(19) United States
(12) Patent Application Publication
(10) Pub. No.: US 2010/0168382 A1
(43) Pub. Date: Jul. 1, 2010

(54) NEUROPLASTIN DERIVED PEPTIDES

(76) Inventor: Vladimir Berezin, (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/664,085

(22) PCT Filed: Jun. 6, 2008
(86) PCT No.: PCT/DK08/50139

§ 371 Date: Dec. 11, 2009

(30) Foreign Application Priority Data
Jun. 12, 2007 (DK) PA 2007 00852

(51) Int. Cl. C07K 14/00; C07K 7/06; C07K 7/08; C07K 16/00

(52) U.S. Cl. 530/324; 530/328; 530/327; 530/326; 530/387.9

(57) ABSTRACT

The present invention relates to peptides derived from neuroplastin which are capable of inducing neurite outgrowth by modulating intracellular calcium concentration and activity of intracellular signalling molecules such as Akt, Erk1/2 and CREB through binding and/or modulation of receptor tyrosine kinases including but not limited to Fibroblast Growth Factor receptors (FGFRs). The peptides are derived from neuroplastin or fragments thereof. The invention further relates to use of said peptides for the production of a medicament for the treatment of different pathological conditions, wherein neuroplastin and/or receptor tyrosine kinases, including but not limited to FGFRs, play a prominent role.

### FIELD OF INVENTION

The present invention relates to peptides derived from neuroplastin which are capable of inducing neurite outgrowth by modulating intracellular calcium concentration and activity of intracellular signalling molecules such as Akt, Erk1/2 and CREB through binding and/or modulation of receptor tyrosine kinases including but not limited to Fibroblast Growth Factor receptors (FGFRs). The peptides are derived from neuroplastin or fragments thereof. The invention further relates to use of said peptides for the production of a medicament for the treatment of different pathological conditions, wherein neuroplastin and/or receptor tyrosine kinases, including but not limited to FGFRs, play a prominent role.

### BACKGROUND OF INVENTION

Neuroplastin is a cell adhesion molecule of the immunoglobulin superfamily that exists in two splice isoforms, neuroplastin 65 and neuroplastin 55. Neuroplastin was reported to play a prominent role in synaptic plasticity processes. The spliced isoform, neuroplastin 65, associates with synapses in an activity-dependent manner and has been shown to play a role for the induction of hippocampal long-term potentiation (LTP) in rodents. Neuroplastin is present in many neuronal cell types of the forebrain and cerebellum, immunoreactive label covers the cell soma, neurites and also puncta in the neuropil are visible. (Bernstein et al., 2007)

Some remarkable species differences in the expression pattern of the neuroplastins between the human and the rodent brain have been noticed. In human brain neuroplastin 65 is prominently present in cerebellum, while neuroplastin 55 is the predominant isoform in mouse and rat cerebellum. The parasagital stripe-like staining seen with neuroplastin 55 in mouse cerebellum is not found in human brain. The results indicate different cellular functions of the molecule in different species. (Bernstein et al., 2006).

The two neuroplastin isoforms arise by alternative splicing from a single gene and contain three (neuroplastin 65) and two (neuroplastin 55) Ig modules, respectively. Both isoforms also contain a single membrane spanning sequence, followed by a short hydrophilic intracellular domain. The neuroplastins are most closely related to the basigin group of immunoglobulin superfamily, which includes basigin and its specific homologs EMMPRIN (CD147), GP42, and 5A11 (Empson et al., 2006).

Recently, promoter polymorphism in the neuroplastin gene has been associated with schizophrenia (Saito et al., 2007).

### SUMMARY OF INVENTION

The present invention describes neuroplastin and fragments thereof being capable of inducing neurite outgrowth by modulating intracellular calcium concentration and the activity of intracellular signalling molecules such as Akt, Erk1/2 and CREB through binding and/or modulation of receptor tyrosine kinases including but not limited to FGFRs.

The invention discloses use of said peptides for induction of differentiation, modulation of proliferation, stimulation of regeneration, neuronal plasticity and survival of cells.

The invention further discloses use of said peptides for the production of a medicament for the treatment of different pathological conditions, wherein neuroplastin and/or receptor tyrosine kinases, including but not limited to FGFRs, plays a role in pathology and/or recovery from disease for example for:
a) treatment of conditions of the central and peripheral nervous system associated with postoperative nerve damage, traumatic nerve damage, impaired myelination of nerve fibers, postischaemic damage, e.g. resulting from a stroke, Parkinson's disease, Alzheimer's disease, Huntington's disease, dementias such as multiinfarct dementia, sclerosis, nerve degeneration associated with diabetes mellitus, disorders affecting the circadian clock or neuro-muscular transmission, and schizophrenia, mood disorders, such as manic depression;
b) treatment of diseases or conditions of the muscles including conditions with impaired function of neuro-muscular connections, such as after organ transplantation, or such as genetic or traumatic atrophic muscle disorders; or for treatment of diseases or conditions of various organs, such as degenerative conditions of the gonads, of the pancreas such as diabetes mellitus type I and II, of the kidney such as nephrosis and of the heart, liver and bowel;
c) promotion of wound-healing;
d) prevention of death of heart muscle cells, such as after acute myocardial infarction;
e) promotion of revascularisation;
f) stimulation of the ability to learn and/or the short and/or long-term memory;
g) treatment of cancer.

It is a further objective of the present invention to provide an antibody capable of selectively binding to an epitope comprising a contiguous amino acid sequence derived from neuroplastin or a fragment, homologue or variant thereof.

### DESCRIPTION OF DRAWINGS

FIG. 1 shows effect of the Enplastin peptide (DPKRNDLRQNPSITWIR) SEQ ID NO: 6 on neurite outgrowth from cerebellar granule neurons (CGN) from postnatal day 7 rats. Cell cultures were grown for 24 h.

FIG. 2 shows effect of the Enplastin peptide (DPKRNDLRQNPSITWIR) SEQ ID NO: 6 on neurite outgrowth hippocampal neurons. Cell cultures were grown for 24 h.

FIG. 3 shows effect of the truncated versions of Enplastin peptide (DPKRNDLRQNPSITWIR), 10 μg/ml each on neurite outgrowth from cerebellar granule neurons (CGN) from postnatal day 7 rats (e and f). Cell cultures were grown for 24 h. (One-way-ANOVA, Dunnett's post test, ** p<0.01, * p<0.05), N1=RNDLRQNPSITWIR SEQ ID NO: 14, N2=NDLRQNPSITWIR SEQ ID NO: 15, N3=LRQNPSITWIR SEQ ID NO: 16, C1=DPKRNDLRQNPSITW SEQ ID NO: 17, C2=DPKRNDLRQNPSI SEQ ID NO: 18, C3=DPKRNDLRQNP SEQ ID NO: 19, C4=DPKRNDLRQ SEQ ID NO: 20.

FIG. 4 shows effect of the alanine substitution of the N1-trancated Enplastin peptide (DPKRNDLRQNPSITWIR) SEQ ID NO: 6 on neurite outgrowth from cerebellar granule neurons (CGN) from postnatal day 7 rats. Cell cultures were grown for 24 h. (One-way-Anova, ** p<0.01), N1=KRNDLRQNPSITWIR SEQ ID NO: 14, A1=ARNDLRQNPSITWIR, SEQ ID NO: 21 A2=KANDLRQNPSITWIR SEQ ID NO: 22, A3=KRADLRQNPSITWIR SEQ ID NO: 23, A4=KRNALRQNPSITWIR SEQ ID NO: 24, A5=KRNDARQNPSITWIR SEQ ID NO: 25, A6=KRNDLAQNPSITWIR SEQ ID NO: 26, A7=KRNDLRANPSITWIR SEQ ID NO: 27, A8=KRNDLRQAPSITWIR SEQ ID NO: 28, A9=KRNDLRQNASITWIR SEQ ID NO: 29, A10=KRNDLRQNPAITWIR SEQ ID NO: 30, A11=KRNDLRQNPSATWIR SEQ ID NO: 31, A12=KRNDLRQNPSIAWIR SEQ ID NO: 32, A13=KRNDLRQNPSITAIR SEQ ID NO: 33, A14=KRNDLRQNPSITWAR SEQ ID NO: 34, A15=KRNDLRQNPSITWIA SEQ ID NO: 35.

FIG. 5 shows effect of the inhibitor of receptor tyrosine kinases (RTK) (lavendustin A) on Enplastin (DPKRNDLRQNPSITWIR) SEQ ID NO: 6 induced on neurite outgrowth from CGN. Lavendustin B is a control, which does not inhibit RTK.

FIG. 6 shows the effect of the inhibitor of FGFR (SU5402) on Enplastin-induced neurite outgrowth (DPKRNDLRQNPSITWIR) SEQ ID NO: 6.

FIG. 7 shows effect of the 1 cds peptide (NRAESFRQLWDGAR) SEQ ID NO: 4 on neurite outgrowth from cerebellar granule neurons (CGN) from postnatal day 7 rats. Cell cultures were grown for 24 h.

FIG. 8 shows effect of the Enplastin peptide (DPKRNDLRQNPSITWIR) SEQ ID NO: 6 on Akt (A) and Erk1/2 B phosforylation in primary hippocampal neurons. Cells were grown for 6 h and then treated with 40 μg/mL Enplastin or 10 nM FGF2 for 10 min (Akt) or 30 min (Erk) and further immunoblotted for phospho-p44/p42 Map Kinase (Thr202/Tyr204) or Ser473-phosphorylated Akt. Representative immunoblots are shown, 1. control. 2. GFGF (10 μM). 3. Enplastin (40 μg/ml).

FIG. 9 shows Effects of the Enplastin peptide (DPKRNDLRQNPSITWIR) SEQ ID NO: 6 and the 1 cds peptide (NRAESFRQLWDGAR) SEQ ID NO: 4 on CREB phosphorylation in primary cultures of hippocampal neurons. Hippocampal neurons were grown in serum-free medium for 4 h and then the cells were treated with 5, 20, 40 and 100 μg/mL Enplastin lfg (green) or 1 cds (black) for 30-min. Results from four independent experiments are expressed as a percentage ±SEM, with the untreated control cultures set at 100%. Phosphorylated CREB was identified by phospho-CREB specific antibodies, and total cell number was estimated by crystal violet staining. *p<0.05 and **p<0.01 when compared with the untreated control (One-way-Anova, Dunnett's post test).

FIG. 10 shows effects of the Enplastin peptide (DPKRNDLRQNPSITWIR) SEQ ID NO: 6 and the 1 cds peptide (NRAESFRQLWDGAR) SEQ ID NO: 4 on Erk phosphorylation in primary cultures of hippocampal neurons. Hippocampal neurons were grown in serum-free medium for 4 h and then the cells were treated with 5, 20, 40 and 100 μg/mL Enplastin (green) or 1 cds (black) for 30-min. Results from four independent experiments are expressed as a percentage ±SEM, with the untreated control cultures set at 100%. Phosphorylated Erk was identified by phospho-Erk specific antibodies, and total cell number was estimated by crystal violet staining. *p<0.05 and **p<0.01 when compared with the untreated control (On-way-Anova, Dannett's post test).

FIG. 11 shows effects of the Enplastin peptide (DPKRNDLRQNPSITWIR) SEQ ID NO: 6 and 1 cds peptide (NRAESFRQLWDGAR) SEQ ID NO: 4 on Akt phosphorylation in primary cultures of hippocampal neurons. Primary cultures of hippocampal neurons were grown in serum-free medium for 4 h and then the cells were treated with 5, 20, 40 and 100 μg/mL Enplastin (green) or 1 cds (black) for 30-min. Results from four independent experiments are expressed as a percentage ±SEM, with the untreated control cultures set at 100%. Phosphorylated Akt was identified by phospho-Akt specific antibodies, and total cell number was estimated by crystal violet staining. *p<0.05 and **p<0.01 when compared with the untreated control (One-way-Anova), Dunnett's post test).

FIG. 12 shows dose-response relationship for the Enplastin peptide SEQ ID NO: 6 (DPKRNDLRQNPSITWIR)-triggered [Ca2+]i rise in hippocampal neurons.

FIG. 13 shows crystal structure of Neuroplastin 55 extracellular domain to 1.9 Å resolution with the narpin peptide (RIVTSEEVIIRDS) SEQ ID NO: 7 highlighted in red.

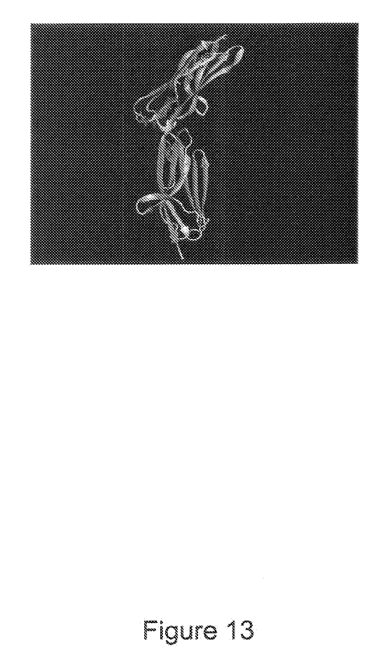

FIG. 14 shows binding of the narpin peptide (RIVTSEEVIIRDS) SEQ ID NO: 7 and its reverse form (SDRIIVEESTVIR1) SEQ ID NO: 36 to the FGFR1.

FIG. 15 shows effect of the narpin peptide (RIVTSEEVIIRDS) SEQ ID NO: 7 on FGFR Phosphorylation. Statistics: One-way-Anova, Newman-Keuls Multiple Comparison Test (*p<0.05, **p<0.01).

FIG. 16 shows effect of the Neuroplastin 55 ectodomain in solution on neurite outgrowth from cerebellar granule neurons (CGN). Statistics: One-way-Anova, Newman-Keuls Multiple Comparison Test (*p<0.05, **p<0.01).

FIG. 17 shows effect of the 2 cd peptide (TKNGVELTATRKNA) SEQ ID NO: 9 on neurite outgrowth from CGN. Statistics: One-way-Anova, Newman-Keuls Multiple Comparison Test (*p<0.05, **p<0.01).

FIG. 18 shows effect of the Neuroplastin55 ectodomain (coated on the slide) on neurite outgrowth from CGN. Statistics: One-way-Anova, Newman-Keuls Multiple Comparison Test (***p<0.001).

FIG. 19 shows effect of the narpin peptide (RIVTSEEVIIRDS) SEQ ID NO: 7 on neurite outgrowth from CGN. Statistics: One-way-Anova, Newman-Keuls Multiple Comparison Test (*p<0.05).

FIG. 20 shows effect of the inhibitor of FGFR, SK5402 on 2 cd-induced neurite outgrowth from hippocampal neurons (*p<0.05, ***p<0.001).

### DETAILED DESCRIPTION OF THE INVENTION

A peptide according to the invention can be neuroplastin or a fragment derived from neuroplastin, or it may be derived from a variant of neuroplastin, such as a natural or recombinant neuroplastin variant, for example a neuroplastin variant produced by alternative splicing, or genetic polymorphism, or any type of recombinant neuroplastin. Examples of peptides of the invention may be the neuroplastin polypeptides identified in the SwissProt database with SwissProt IDs: Q9Y639-2 and P97546-2.

A peptide according to the invention is a peptide which is capable of inducing neurite outgrowth and modulating intracellular calcium concentration and the activity of intracellular signalling molecules such as Akt, Erk1/2 and CREB through binding and/or activation of receptor tyrosine kinases including but not limited to FGFRs. By the terms “modulation” or “modulating” are meant a change, such as an inhibition or stimulation.

### Amino Acid Sequence

Peptides according to the invention comprise neuroplastin or a fragment thereof which comprises a contiguous amino acid sequence derived from neuroplastin or a fragment, homologue or variant thereof.

In a preferred embodiment the peptides according to the invention may comprise a contiguous amino acid sequence which is derived from neuroplastin. Accordingly, in this embodiment the amino acid sequence according to the invention may be selected from the following amino acid sequences:

### Neuroplastin:

SEQ ID NO: 1

MSGSSLPSALALSLLLVSGSLLPGPGAAQNAGFVKS
PMSETKLTGDAFELYCDVVGSPTPEIQWWYAEVNRA
ESFRQLWDGARKRRVTVNTAYGSNGVSVLRITRLTL
EDSGTYECRASNDPKRNDLRQNPSITWIRAQATISVL
QKPRIVTSEEVIIRDSPVLPVTLQCNLTSSSHTLTYSY
WTKNGVELSATRKNASNMEYRINKPRAEDSGEYHC
VYHFVSAPKANATIEVKAAPDITGHKRSENKNEGQD
ATMYCKSVGYPHPDWIWRKKENGMPMDIVNTSGRF
FIINKENYTELNIVNLQITEDPGEYECNATNAIGSASV
VTVLRVRSHLAPLWPFLGILAEIIILVVIIVVYEKRKRPD
EVPDDDEPAGPMKTNSTNNHKDKNLRQRNTN

### Fragments of SEQ ID NO:1:

| | | |
|---|---|---|
| NRPL1ab_s: | TKLTGDAFEL | SEQ ID NO: 2 |
| NRPL1bc: | DVVGSPTPEIQ | SEQ ID NO: 3 |
| NRPL1cd_s: | NRAESFRQLWDGAR | SEQ ID NO: 4 |
| NRPL1dd: | RRVTVNTAYGSNG | SEQ ID NO: 5 |
| Enplastin: | DPKRNDLRQNPSITWIR | SEQ ID NO: 6 |
| Narpin: | RIVTSEEVIIRDS | SEQ ID NO: 7 |
| NRPL2bc_s: | NLTSSSHTLMYS | SEQ ID NO: 8 |
| NRPL2cd: | TKNGVELTATRKNA | SEQ ID NO: 9 |
| NRPL2de: | KNASNMEYRINKP | SEQ ID NO: 10 |
| NRPL2ef: | NKPRAEDSGE | SEQ ID NO: 11 |
| NRPL2fg: | VYHFVSAPKANAT | SEQ ID NO: 12 |
| NRPL3de: | INKENYTELN. | SEQ ID NO: 13 |

In the present context the standard one-letter code for amino acid residues as well as the standard three-letter code are applied. Abbreviations for amino acids are in accordance with the recommendations in the IUPAC-IUB Joint Commission on Biochemical Nomenclature Eur. J. Biochem, 1984, vol. 184, pp 9-37. Throughout the description and claims either the three letter code or the one letter code for natural amino acids are used. Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

Where nothing is specified it is to be understood that the C-terminal amino acid of a peptide for use according to the invention exists as the free carboxylic acid, this may also be specified as “—OH”. However, the C-terminal amino acid of a peptide for use according to the invention may be the amidated derivative, which is indicated as “—NH₂”. Where nothing else is stated the N-terminal amino acid of a polypeptide comprises a free amino-group, this may also be specified as “H—”.

A peptide, fragment, homologue or variant thereof according to the invention can also comprise one or several unnatural amino acids.

A preferred peptide according to the invention is neuroplastin with SEQ ID NO:1. Another preferred peptide according to the invention is an isolated contiguous peptide sequence which comprises at most 25 amino acid residues of SEQ ID NO:1. In one embodiment the length of the amino acid sequence of a peptide may be from 3 to 9 amino acid residues, such as for example 4, 5, 6, 7, or 8, amino acid residues. In another embodiment, the length of the amino acid sequence of a peptide may be from 10-25 amino acid residues, such as for example 11, 12, 13, 14, 15, 16, 17, 18, 19, 20, 21, 22, 23 or 24 amino acid residues. It is understood that all peptides according to the invention comprise at least one amino acid sequence selected from any of the sequences SEQ ID NOs: 1-13 or a fragment, variant or homologue thereof.

Thus, some embodiments of the invention may relate to a peptide comprising a fragment of a sequence selected from SEQ ID NOs:1-13. Another embodiment may relate to variants of SEQ ID NOs:1-13. A further embodiment may relate to homologues of SEQ ID NOs: 1-13.

A variant according to the invention of an amino acid sequence selected from the sequences SEQ ID NOs: 1-13 may be
i) an amino acid sequence which has at least 75% identity with a selected sequence, such as 76-80% identity, for example 81-85% identity, such as 86-90% identity, for example 91-95% identity, such as 96-99% identity, wherein the identity is defined as a percentage of identical amino acids in said sequence when it is collated with the selected sequence. The identity between amino acid sequences may be calculated using well known algorithms such as BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, or BLOSUM 90;
ii) an amino acid sequence which has at least 75% positive amino acid matches with a selected sequence, such as 76-80% positive amino acid matches, for example 81-85% positive amino acid matches, such as 86-90% positive amino acid matches, for example 91-95% positive amino acid matches, such as 96-99% positive amino acid matches, wherein the positive amino acid match is defined as the presence at the same position in two compared sequences of amino acid residues which has similar physical and/or chemical properties. Preferred positive amino acid matches of the present invention are K to R, E to D, L to M, Q to E, Ito V, Ito L, A to S, Y to W, K to Q, S to T, N to S and Q to R;
iii) an amino acid sequence which is identical to a selected sequence, or it has at least 75% identity with said sequence such as 76-80% identity, for example 81-85% identity, such as 86-90% identity, for example 91-95% identity, such as 96-99% identity, or has at least 75% positive amino acid matches with the selected sequence, such as 76-80% positive amino acid matches, for example 81-85% positive amino acid matches, such as 86-90% positive amino acid matches, for example 91-95% positive amino acid matches, such as 96-99% positive amino acid matches, and comprises other chemical moieties, e.g. phosphoryl, sulphur, acetyl, glycosyl moieties.

The term “variant of a peptide sequence” also means that the peptide sequence may be modified, for example by substitution of one or more of the amino acid residues. Both L-amino acids and D-amino acids may be used. Other modification may comprise derivatives such as esters, sugars, etc., for example methyl and acetyl esters.

In another aspect, variants of the amino acid sequences according to the invention may comprise, within the same variant, or fragments thereof or among different variants, or fragments thereof, at least one substitution, such as a plurality of substitutions introduced independently of one another. Variants of the complex, or fragments thereof may thus comprise conservative substitutions independently of one another, wherein at least one glycine (Gly) of said variant, or fragments thereof is substituted with an amino acid selected from the group of amino acids consisting of Ala, Val, Leu, and Ile, and independently thereof, variants, or fragments thereof, wherein at least one alanine (Ala) of said variants, or fragments thereof is substituted with an amino acid selected from the group of amino acids consisting of Gly, Val, Leu, and Ile, and independently thereof, variants, or fragments thereof, wherein at least one valine (Val) of said variant, or fragments thereof is substituted with an amino acid selected from the group of amino acids consisting of Gly, Ala, Leu, and Ile, and independently thereof, variants, or fragments thereof, wherein at least one leucine (Leu) of said variant, or fragments thereof is substituted with an amino acid selected from the group of amino acids consisting of Gly, Ala, Val, and Ile, and independently thereof, variants, or fragments thereof, wherein at least one isoleucine (Ile) of said variants, or fragments thereof is substituted with an amino acid selected from the group of amino acids consisting of Gly, Ala, Val and Leu, and independently thereof, variants, or fragments thereof wherein at least one aspartic acids (Asp) of said variant, or fragments thereof is substituted with an amino acid selected from the group of amino acids consisting of Glu, Asn, and Gln, and independently thereof, variants, or fragments thereof, wherein at least one aspargine (Asn) of said variants, or fragments thereof is substituted with an amino acid selected from the group of amino acids consisting of Asp, Glu, and Gln, and independently thereof, variants, or fragments thereof, wherein at least one glutamine (Gln) of said variants, or fragments thereof is substituted with an amino acid selected from the group of amino acids consisting of Asp, Glu, and Asn, and wherein at least one phenylalanine (Phe) of said variants, or fragments thereof is substituted with an amino acid selected from the group of amino acids consisting of Tyr, Trp, His, Pro, and preferably selected from the group of amino acids consisting of Tyr and Trp, and independently thereof, variants, or fragments thereof, wherein at least one tyrosine (Tyr) of said variants, or fragments thereof is substituted with an amino acid selected from the group of amino acids consisting of Phe, Trp, His, Pro, preferably an amino acid selected from the group of amino acids consisting of Phe and Trp, and independently thereof, variants, or fragments thereof, wherein at least one arginine (Arg) of said fragment is substituted with an amino acid selected from the group of amino acids consisting of Lys and His, and independently thereof, variants, or fragments thereof, wherein at least one lysine (Lys) of said variants, or fragments thereof is substituted with an amino acid selected from the group of amino acids consisting of Arg and His, and independently thereof, variants, or fragments thereof, and independently thereof, variants, or fragments thereof, and wherein at least one proline (Pro) of said variants, or fragments thereof is substituted with an amino acid selected from the group of amino acids consisting of Phe, Tyr, Trp, and His, and independently thereof, variants, or fragments thereof, wherein at least one cysteine (Cys) of said variants, or fragments thereof is substituted with an amino acid selected from the group of amino acids consisting of Asp, Glu, Lys, Arg, His, Asn, Gln, Ser, Thr, and Tyr.

It thus follows from the above that the same variant of a peptide fragment, or fragment of said variant may comprise more than one conservative amino acid substitution from more than one group of conservative amino acids as defined herein above. The term “conservative amino acid substitution” is used synonymously herein with the term “homologous amino acid substitution”.

The groups of conservative amino acids are as the following:

A, G (neutral, weakly hydrophobic),
Q, N, S, T (hydrophilic, non-charged)
E, D (hydrophilic, acidic)
H, K, R (hydrophilic, basic)
L, P, I, V, M, F, Y, W (hydrophobic, aromatic)
C (cross-link forming)

Conservative substitutions may be introduced in any position of a preferred predetermined peptide for use according to the invention or fragment thereof. It may however also be desirable to introduce non-conservative substitutions, particularly, but not limited to, a non-conservative substitution in any one or more positions.

A non-conservative substitution leading to the formation of a variant fragment of the peptide for use according to the invention would for example differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on peptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like.

Both fragments and variants of amino acid sequences according to the invention are the functional equivalents of said sequences.

By the term “functional equivalent” of an amino acid sequence is in the present context meant a molecule which meets the criteria for a variant or a fragment of said amino acid sequence described above and which is capable of one or more functional activities of said sequence or a compound comprising said sequence. In a preferred embodiment the functional equivalent of an amino acid sequence according to the invention is capable of binding and modulating activity of receptor tyrosine kinases, including but not limited to FGFRs.

The invention relates both to isolated peptides according to the invention and fusion proteins comprising peptides according to the invention.

In one embodiment, the peptide according to the invention is an isolated peptide. By the term “isolated peptide” is meant that the peptide according to the invention is an individual compound and not a part of another compound, such as for example a polypeptide comprising more then 25 amino acid residues. The isolated peptide may be produced by use of any recombinant technology methods or chemical synthesis and separated from other compounds, or it may be separated from a longer polypeptide or protein by a method of enzymatic or chemical cleavage and further separated from other protein fragments.

An isolated peptide according to the invention may in one embodiment comprise neuroplastin with SEQ ID NO:1. An isolated peptide according to the invention may in another embodiment comprise a fragment of neuroplastin which comprises a contiguous amino acid sequence derived from neuroplastin, selected from SEQ ID NOs:2-13 or a fragment or variant thereof. In another embodiment the isolated peptide may consist of one or more of the sequences SEQ ID NOs:1-13.

### Production of Peptide Sequences

The peptide sequences of the present invention may be prepared by any conventional synthetic methods, recombinant DNA technologies, enzymatic cleavage of full-length proteins which the peptide sequences are derived from, or a combination of said methods.

### Recombinant Preparation

Thus, in one embodiment the peptides of the invention are produced by use of recombinant DNA technologies.

The DNA sequence encoding a peptide or the corresponding full-length protein the peptide originates from may be prepared synthetically by established standard methods, e.g. the phosphoamidine method described by Beaucage and Caruthers, 1981, Tetrahedron Lett. 22:1859-1869, or the method described by Matthes et al., 1984, EMBO J. 3:801-805. According to the phosphoamidine method, oligonucleotides are synthesised, e.g. in an automatic DNA synthesiser, purified, annealed, ligated and cloned in suitable vectors.

The DNA sequence encoding a peptide may also be prepared by fragmentation of the DNA sequences encoding the corresponding full-length protein of peptide origin, using DNAase I according to a standard protocol (Sambrook et al., Molecular cloning: A Laboratory manual. 2 rd ed., CSHL Press, Cold Spring Harbor, N.Y., 1989). The present invention relates to full-length proteins selected from the groups of proteins identified above. The DNA encoding the full-length proteins of the invention may alternatively be fragmented using specific restriction endonucleases. The fragments of DNA are further purified using standard procedures described in Sambrook et al., Molecular cloning: A Laboratory manual. 2 rd ed., CSHL Press, Cold Spring Harbor, N.Y., 1989.

The DNA sequence encoding a full-length protein may also be of genomic or cDNA origin, for instance obtained by preparing a genomic or cDNA library and screening for DNA sequences coding for all or part of the full-length protein by hybridisation using synthetic oligonucleotide probes in accordance with standard techniques (cf. Sambrook et al., Molecular Cloning: A Laboratory Manual, 2nd Ed., Cold Spring Harbor, 1989). The DNA sequence may also be prepared by polymerase chain reaction using specific primers, for instance as described in U.S. Pat. No. 4,683,202 or Saiki et al., 1988, Science 239:487-491.

The DNA sequence is then inserted into a recombinant expression vector, which may be any vector, which may conveniently be subjected to recombinant DNA procedures. The choice of vector will often depend on the host cell into which it is to be introduced. Thus, the vector may be an autonomously replicating vector, i.e. a vector that exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

In the vector, the DNA sequence encoding a peptide or a full-length protein should be operably connected to a suitable promoter sequence. The promoter may be any DNA sequence, which shows transcriptional activity in the host cell of choice and may be derived from genes encoding proteins either homologous or heterologous to the host cell. Examples of suitable promoters for directing the transcription of the coding DNA sequence in mammalian cells are the SV 40 promoter (Subramani et al., 1981, Mol. Cell. Biol. 1:854-864), the MT-1 (metallothionein gene) promoter (Palmiter et al., 1983, Science 222: 809-814) or the adenovirus 2 major late promoter. A suitable promoter for use in insect cells is the polyhedrin promoter (Vasuvedan et al., 1992, FEBS Lett. 311:7-11). Suitable promoters for use in yeast host cells include promoters from yeast glycolytic genes (Hitzeman et al., 1980, J. Biol. Chem. 255:12073-12080; Alber and Kawasaki, 1982, J. Mol. Appl. Gen. 1: 419-434) or alcohol dehydrogenase genes (Young et al., 1982, in Genetic Engineering of Microorganisms for Chemicals, Hollaender et al, eds., Plenum Press, New York), or the TPI1 (U.S. Pat. No. 4,599,311) or ADH2-4-c (Russell et al., 1983, Nature 304:652-654) promoters. Suitable promoters for use in filamentous fungus host cells are, for instance, the ADH3 promoter (McKnight et al., 1985, EMBO J. 4:2093-2099) or the tpiA promoter.

The coding DNA sequence may also be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) promoters. The vector may further comprise elements such as polyadenylation signals (e.g. from SV 40 or the adenovirus 5 E1b region), transcriptional enhancer sequences (e.g. the SV 40 enhancer) and translational enhancer sequences (e.g. the ones encoding adenovirus VA RNAs).

The recombinant expression vector may further comprise a DNA sequence enabling the vector to replicate in the host cell in question. An example of such a sequence (when the host cell is a mammalian cell) is the SV 40 origin of replication. The vector may also comprise a selectable marker, e.g. a gene the product of which complements a defect in the host cell, such as the gene coding for dihydrofolate reductase (DHFR) or one which confers resistance to a drug, e.g. neomycin, hydromycin or methotrexate.

The procedures used to ligate the DNA sequences coding the peptides or full-length proteins, the promoter and the terminator, respectively, and to insert them into suitable vectors containing the information necessary for replication, are well known to persons skilled in the art (cf., for instance, Sambrook et al., op.cit.).

To obtain recombinant peptides of the invention the coding DNA sequences may be usefully fused with a second peptide coding sequence and a protease cleavage site coding sequence, giving a DNA construct encoding the fusion protein, wherein the protease cleavage site coding sequence positioned between the HBP fragment and second peptide coding DNA, inserted into a recombinant expression vector, and expressed in recombinant host cells. In one embodiment, said second peptide selected from, but not limited by the group comprising glutathion-5-reductase, calf thymosin, bacterial thioredoxin or human ubiquitin natural or synthetic variants, or peptides thereof. In another embodiment, a peptide sequence comprising a protease cleavage site may be the Factor Xa, with the amino acid sequence IEGR, enterokinase, with the amino acid sequence DDDDK, thrombin, with the amino acid sequence LVPR/GS, or Acharombacter lyticus, with the amino acid sequence XKX, cleavage site.

The host cell into which the expression vector is introduced may be any cell which is capable of expression of the peptides or full-length proteins, and is preferably a eukaryotic cell, such as invertebrate (insect) cells or vertebrate cells, e.g. Xenopus Iaevis oocytes or mammalian cells, in particular insect and mammalian cells. Examples of suitable mammalian cell lines are the HEK293 (ATCC CRL-1573), COS (ATCC CRL-1650), BHK (ATCC CRL-1632, ATCC CCL-10) or CHO (ATCC CCL-61) cell lines. Methods of transfecting mammalian cells and expressing DNA sequences introduced in the cells are described in e.g. Kaufman and Sharp, J. Mol. Biol. 159, 1982, pp. 601-621; Southern and Berg, 1982, J. Mol. Appl. Genet. 1:327-341; Loyter et al., 1982, Proc. Natl. Acad. Sci. USA 79: 422-426; Wigler et al., 1978, Cell 14:725; Corsaro and Pearson, 1981, in Somatic Cell Genetics 7, p. 603; Graham and van der Eb, 1973, Virol. 52:456; and Neumann et al., 1982, EMBO J. 1:841-845.

Alternatively, fungal cells (including yeast cells) may be used as host cells. Examples of suitable yeast cells include cells of Saccharomyces spp. or Schizosaccharomyces spp., in particular strains of Saccharomyces cerevisiae. Examples of other fungal cells are cells of filamentous fungi, e.g. Aspergillus spp. or Neurospora spp., in particular strains of Aspergillus oryzae or Aspergillus niger. The use of Aspergillus spp. for the expression of proteins is described in, e.g., EP 238 023.

The medium used to culture the cells may be any conventional medium suitable for growing mammalian cells, such as a serum-containing or serum-free medium containing appropriate supplements, or a suitable medium for growing insect, yeast or fungal cells. Suitable media are available from commercial suppliers or may be prepared according to published recipes (e.g. in catalogues of the American Type Culture Collection).

The peptides or full-length proteins recombinantly produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. HPLC, ion exchange chromatography, affinity chromatography, or the like.

### Synthetic Preparation

The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

Peptides may for example be synthesised by using Fmoc chemistry and with Acm-protected cysteins. After purification by reversed phase HPLC, peptides may be further processed to obtain for example cyclic or C- or N-terminal modified isoforms. The methods for cyclization and terminal modification are well-known in the art and described in detail in the above-cited manuals.

In a preferred embodiment the peptide sequences of the invention are produced synthetically, in particular, by the Sequence Assisted Peptide Synthesis (SAPS) method.

Peptides may be synthesised either batchwise in a polyethylene vessel equipped with a polypropylene filter for filtration or in the continuous-flow version of the polyamide solid-phase method (Dryland, A. and Sheppard, R. C., (1986) J. Chem. Soc. Perkin Trans. I, 125-137.) on a fully automated peptide synthesiser using 9-fluorenylmethyloxycarbonyl (Fmoc) or tert.-Butyloxycarbonyl, (Boc) as N-a-amino protecting group and suitable common protection groups for side-chain functionality's.

### Medicament

It is an objective of the invention to provide a compound capable of inducing neurite outgrowth by modulating the activity of receptor tyrosine kinases including but not limited to FGFRs and/or the kinases Akt, Erk1/2 and the transcription factor CREB, said compound being concerned by the invention as a medicament for the treatment of diseases, wherein modulating the activity of FGFRs and/or the kinases Akt, Erk1/2 and the transcription factor CREB may be considered as an essential condition for curing.

Accordingly, the invention relates to the use of one or more of the peptides comprising a sequence corresponding to neuroplastin or a fragment thereof or a variant for the manufacture of a medicament.

In one embodiment the medicament of the invention comprises at least one of the amino acid sequences set forth in SEQ ID NOS: 1-13 or fragments or variants or homologues of said sequences, or fragments or variants of said homologues. In another embodiment the medicament of the invention comprises an antibody capable of binding to an epitope comprising neuroplastin or a fragment thereof or a fragment or variant of said antibody.

The medicament may in one aspect prevent death of cells in vitro or in vivo, wherein the composition is administered to a subject, in vitro or in vivo in an effective amount of one or more of the compounds described above or a composition as described below.

The medicament of the invention comprises an effective amount of one or more of the compounds as defined above, or a composition comprising compound as defined above, in combination with pharmaceutically acceptable additives. Such medicament may suitably be formulated for oral, percutaneous, intramuscular, intravenous, intracranial, intrathecal, intracerebroventricular, intranasal or pulmonal administration.

Strategies in formulation development of medicaments and compositions based on the peptides of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 5000 μg per kilo body weight. Using monomeric forms of the compounds, the suitable dosages are often in the range of from 0.1 μg to 5000 μg per kilo body weight, such as in the range of from about 0.1 μg to 3000 μg per kilo body weight, and especially in the range of from about 0.1 μg to 1000 μg per kilo body weight. Using multimeric forms of the compounds, the suitable dosages are often in the range of from 0.1 μg to 1000 μg per kilo body weight, such as in the range of from about 0.1 μg to 750 μg per kilo body weight, and especially in the range of from about 0.1 μg to 500 μg per kilo body weight such as in the range of from about 0.1 μg to 250 μg per kilo body weight. In particular when administering nasally smaller dosages are used than when administering by other routes. Administration may be performed once or may be followed by subsequent administrations. The dosage will also depend on the route of administration and will vary with the age and weight of the subject to be treated. A preferred dosage of multimeric forms would be in the interval 1 mg to 70 mg per 70 kg body weight.

For most indications a localised or substantially localised application is preferred.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promotes delivery of the active substance to its target.

In many instances, it will be necessary to administrate the formulation multiple times. Administration may be a continuous infusion, such as intraventricular infusion or administration in more doses such as more times a day, daily, more times a week, weekly, etc. It is preferred that administration of the medicament is initiated before or shortly after the individual has been subjected to the factor(s) that may lead to cell death. Preferably the medicament is administered within 8 hours from the factor onset, such as within 5 hours from the factor onset. Many of the compounds exhibit a long term effect whereby administration of the compounds may be conducted with long intervals, such as 1 week or 2 weeks.

In connection with the use in nerve guides, the administration may be continuous or in small portions based upon controlled release of the active compound(s). Furthermore, precursors may be used to control the rate of release and/or site of release. Other kinds of implants and well as oral administration may similarly be based upon controlled release and/or the use of precursors.

As discussed above, the present invention relates to treatment of individuals for inducing differentiation, modulating proliferation, stimulate regeneration, neuronal plasticity and survival of cells in vitro or in vivo, the treatment involving administering an effective amount of one or more compounds as defined above.

Another strategy for administration is to implant or inject cells capable of expressing and secreting the compound in question. Thereby the compound may be produced at the location where it is going to act.

### Treatment

Treatment according to the invention is in one embodiment useful for inducing differentiation, modulating proliferation, stimulating regeneration, neuronal plasticity and survival of cells, for example cells being implanted or transplanted.

In further embodiment the treatment may be for stimulation of survival of cells which are at risk of dying due to a variety of factors, such as traumas and injuries, acute diseases, chronic diseases and/or disorders, in particular degenerative diseases normally leading to cell death, other external factors, such as medical and/or surgical treatments and/or diagnostic methods that may cause formation of free radicals or otherwise have cytotoxic effects, such as X-rays and chemotherapy. In relation to chemotherapy peptides according to the invention are useful in cancer treatment.

Thus, the treatment comprises treatment and/or prophylaxis of cell death in relation to diseases or conditions of the central and peripheral nervous system, such as postoperative nerve damage, traumatic nerve damage, e.g. resulting from spinal cord injury, impaired myelination of nerve fibers, postischaemic damage, e.g. resulting from a stroke, multiinfarct dementia, multiple sclerosis, nerve degeneration associated with diabetes mellitus, neuro-muscular degeneration, schizophrenia, Alzheimer's disease, Parkinson's disease, or Huntington's disease.

Also, in relation to diseases or conditions of the muscles including conditions with impaired function of neuro-muscular connections, such as genetic or traumatic atrophic muscle disorders; or for the treatment of diseases or conditions of various organs, such as degenerative conditions of the gonads, of the pancreas, such as diabetes mellitus type I and II, of the kidney, such as nephrosis the compounds according to the invention may be used for inducing differentiation, modulating proliferation, stimulate regeneration, neuronal plasticity and survival , i.e. stimulating survival.

Furthermore, the treatment may be for preventing cell death of heart muscle cells, such as after acute myocardial infarction, in order to induce angiogenesis. Furthermore, in one embodiment the treatment is for the stimulation of the survival of heart muscle cells, such as survival after acute myocardial infarction. In another aspect the treatment is for revascularisation, such as after injuries.

It is also within the scope of the invention a use of the peptides for the promotion of wound-healing. The present peptides are capable of stimulating angiogenesis and thereby they can promote the wound healing process.

The invention further discloses a use of peptides in the treatment of cancer. Regulation of activation of receptor tyrosine kinases is important for tumor agiogenesis, proliferation and spreading.

In yet a further embodiment a use of the peptides is for the stimulation of the ability to learn and/or of the short and/or long term memory, as FGFR activity is important for differentiation of neural cells.

In still another embodiment a peptide for use according to the invention is for the treatment of body damages due to alcohol consumption. Developmental malformations of foetuses, long-term neurobehavioral alterations, alcoholic liver disease are particularly concerned.

Therapeutic treatment of prion diseases including using a peptide is still another embodiment of the invention.

In particular the use according to the invention of a peptide may be for the treatment of clinical conditions, such as neoplasms such as malignant neoplasms, benign neoplasms, carcinoma in situ and neoplasms of uncertain behavior, cancer in breast, thyroidal, pancreas, brain, lung, kidney, prostate, liver, heart, skin, blood organ, muscles (sarcoma), cancers with dysfunction and/or over- or under-expression of specific receptors and/or expression of mutated receptors or associated with soluble receptors, such as but not limited to Erb-receptors and FGF-receptors, diseases of endocrine glands, such as diabetes mellitus I and II, pituitary gland tumor, psychoses, such as senile and presenile organic psychotic conditions, alcoholic psychoses, drug psychoses, transient organic psychotic conditions, Alzheimer's disease, cerebral lipidoses, epilepsy, general paresis [syphilis], hepatolenticular degeneration, Huntington's chorea, Jakob-Creutzfeldt disease, multiple sclerosis, Pick's disease of the brain, polyareriti nodosa, syphilis, schizophrenic disorders, affective psychoses, neurotic disorders, personality disorders, including character neurosis, nonpsychotic personality disorder associated with organic brain syndromes, paranoid personality disorder, fanatic personality, paranoid personality (disorder), paranoid traits, sexual deviations and disorders or dysfunctions (including reduced sexual drive for what ever reason), mental retardation, disease in the nervesystem and sense organs, such as affecting sight, hearing, smell, feeling, tasting, cognitive anomalies after disease, injury (e.g. after trauma, surgical procedure, and violence), inflammatory disease of the central nervous system, such as meningitis, encephalitis, cerebral degenerations such as Alzheimer's disease, Pick's disease, senile degeneration of brain, senility NOS, communicating hydrocephalus, obstructive hydrocephalus, Parkinson's disease including other extra pyramidal disease and abnormal movement disorders, spinocerebellar disease, cerebellar ataxia, Marie's Sanger-Brown, Dyssynergia cerebellaris myoclonica, primary cerebellar degeneration, such as spinal muscular atrophy, familial, juvenile, adult spinal muscular atrophy, motor neuron disease, amyotrophic lateral sclerosis, motor neuron disease, progressive bulbar palsy, pseudobulbar palsy, primary lateral sclerosis, other anterior horn cell diseases, anterior horn cell disease, unspecified, other diseases of spinal cord, syringomyelia and syringobulbia, vascular myelopathies, acute infarction of spinal cord (embolic) (nonembolic), arterial thrombosis of spinal cord, edema of spinal cord, hematomyelia, subacute necrotic myelopathy, subacute combined degeneration of spinal cord in diseases classified elsewhere, myelopathy, drug-induced, radiation-induced myelitis, disorders of the autonomic nervous system, disorders of peripheral autonomic, sympathetic, parasympathetic, or vegetative system, familial dysautonomia [Riley-Day syndrome], idiopathic peripheral autonomic neuropathy, carotid sinus syncope or syndrome, cervical sympathetic dystrophy or paralysis. peripheral autonomic neuropathy in disorders classified elsewhere, amyloidosis, diseases of the peripheral nerve system, brachial plexus lesions, cervical rib syndrome, costoclavicular syndrome, scalenus anticus syndrome, thoracic outlet syndrome, brachial neuritis or radiculitis NOS, including in newborn. Inflammatory and toxic neuropathy, including acute infective polyneuritis, Guillain-Barre syndrome, Postinfectious polyneuritis, polyneuropathy in collagen vascular disease, disorders of the globe including disorders affecting multiple structures of eye, such as purulent endophthalmitis, diseases of the ear and mastoid process, chronic rheumatic heart disease, ischaemic heart disease, arrhythmia, diseases in the pulmonary system, respiratory system, sensoring e.g. oxygene, astma, abnormality of organs and soft tissues in newborn, including in the nerve system, complications of the administration of anesthetic or other sedation in labor and delivery, diseases in the skin including infection, insufficient circulation problem, burn injury and other mechanic and/or physical injuries, injuries, including after surgery, crushing injury, burns. Injuries to nerves and spinal cord, including division of nerve, lesion in continuity (with or without open wound), traumatic neuroma (with or without open wound), traumatic transient paralysis (with or without open wound), accidental puncture or laceration during medical procedure, injury to optic nerve and pathways, optic nerve injury, second cranial nerve, injury to optic chiasm, injury to optic pathways, injury to visual cortex, unspecified blindness, injury to other cranial nerve(s), injury to other and unspecified nerves, poisoning by drugs, medicinal and biological substances, genetic or traumatic atrophic muscle disorders; or for the treatment of diseases or conditions of various organs, such as degenerative conditions of the gonads, of the pancreas, such as diabetes mellitus type I and II, of the kidney, such as nephrosis. Scrapie, Creutzfeldt-Jakob disease, Gerstmann-Straussler-Sheinker (GSS) disease; pain syndrome, encephalitis, drug/alcohol abuse, anxiety, postoperative nerve damage, peri-operative ischemia, inflammatory disorders with tissue damage, either by affecting the infections agent or protecting the tissue, HIV, hepatitis, and following symptoms, autoimmune disorders, such as rheumatoid arthritis, SLE, ALS, and MS. Anti-inflammatory effects, asthma and other allergic reactions, acute myocardial infarction, and other related disorders or sequel from AMI, metabolic disorders, such as obscenity lipid disorders (e.g. hyper cholestorolamia, artheslerosis, disorders of amino-acid transport and metabolism, disorders of purine and pyrimidine metabolism and gout, bone disorders, such as fracture, osteoporosis, osteo arthritis (OA), Atrophic dermatitis, psoriasis, infection cased disorders, stem cell protection or maturation in vivo or in vitro.

### Antibody

It is an objective of the present invention to provide the use of an antibody, antigen binding fragment or recombinant protein thereof capable of selectively binding to an epitope comprising a contiguous amino acid sequence derived from neuroplastin or a fragment, homologue or variant thereof. The invention relates to any antibody capable of selectively binding to an epitope comprising a contiguous amino acid sequence derived from neuroplastin, selected from any of the sequences set forth in SEQ ID NOS: 1-13, or a fragment or variant of said sequence.

By the term “epitope” is meant the specific group of atoms (on an antigen molecule) that is recognized by (that antigen's) antibodies. The term “epitope” is the equivalent to the term “antigenic determinant”. The epitope may comprise 3 or more amino acid residues, such as for example 4, 5, 6, 7, 8 amino acid residues, located in close proximity, such as within a contiguous amino acid sequence, or located in distant parts of the amino acid sequence of an antigen, but due to protein folding have been approached to each other.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Novotny J, & Haber E. Proc Natl Acad Sci U S A. 82(14):4592-6, 1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv, Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In the context of both the therapeutic and screening methods described below, preferred embodiments are the use of an antibody or fragment thereof that is immunospecific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

The term “antibody fragment” is used herein interchangeably with the term “antigen binding fragment”.

Antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it is has similar or immunological properties relative to antibody that binds with specificity to an epitope comprising a peptide sequence selected from any of the sequences identified herein as SEQ ID NOs: 1-13, or a fragment of said sequences. Thus, in context of the present invention the term “antibody fragment” is identical to term “antigen binding fragment”.

Antibody fragments retain some ability to selectively bind with its antigen or receptor. Some types of antibody fragments are defined as follows:
(1) Fab is the fragment that contains a monovalent antigen-binding fragment of an antibody molecule. A Fab fragment can be produced by digestion of whole antibody with the enzyme papain to yield an intact light chain and a portion of one heavy chain.
(2) Fab′ is the fragment of an antibody molecule can be obtained by treating whole antibody with pepsin, followed by reduction, to yield an intact light chain and a portion of the heavy chain. Two Fab′ fragments are obtained per antibody molecule.

Fab′ fragments differ from Fab fragments by the addition of a few residues at the carboxyl terminus of the heavy chain CH1 domain including one or more cysteines from the antibody hinge region.
(3) (Fab′)₂ is the fragment of an antibody that can be obtained by treating whole antibody with the enzyme pepsin without subsequent reduction.
(4) F(ab′)₂ is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.
(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies 113: 269-315 Rosenburg and Moore eds. Springer-Verlag, NY, 1994.

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

The invention also contemplates multivalent antibodies having at least two binding domains. The binding domains may have specificity for the same ligand or for different ligands. In one embodiment the multispecific molecule is a bispecific antibody (BsAb), which carries at least two different binding domains, at least one of which is of antibody origin. Multivalent antibodies may be produced by a number of methods. Various methods for preparing bi- or multivalent antibodies are for example described in U.S. Pat. Nos. 5,260,203; 5,455,030; 4,881,175; 5,132,405; 5,091,513; 5,476,786; 5,013,653; 5,258,498; and 5,482,858.

The invention contemplate both polyclonal and monoclonal antibody, antigen binding fragments and recombinant proteins thereof which are capable of binding an epitope according to the invention.

The preparation of polyclonal antibodies is well-known to those skilled in the art. See, for example, Green et al. 1992. Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1, which are hereby incorporated by reference.

The preparation of monoclonal antibodies likewise is conventional. See, for example, Kohler & Milstein, Nature, 256:495-7 (1975); Coligan, et al., sections 2.5.1-2.6.7; and Harlow, et al., in: Antibodies: A Laboratory Manual, page 726, Cold Spring Harbor Pub. (1988), Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG). In: Methods in Molecular Biology, 1992, 10:79-104, Humana Press, NY.

Methods of in vitro and in vivo manipulation of monoclonal antibodies are well known to those skilled in the art. For example, the monoclonal antibodies to be used in accordance with the present invention may be made by the hybridoma method first described by Kohler and Milstein, 1975, Nature 256, 495-7, or may be made by recombinant methods, e.g., as described in U.S. Pat. No. 4,816,567. The monoclonal antibodies for use with the present invention may also be isolated from phage antibody libraries using the techniques described in Clackson et al., 1991, Nature 352: 624-628, as well as in Marks et al., 1991, J Mol Biol 222: 581-597. Another method involves humanizing a monoclonal antibody by recombinant means to generate antibodies containing human specific and recognizable sequences. See, for review, Holmes, et al., 1997, J Immunol 158:2192-2201 and Vaswani, et al., 1998, Annals Allergy, Asthma & Immunol 81:105-115.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al., 1984, Proc Natl Acad Sci 81: 6851-6855.

Methods of making antibody fragments are also known in the art (see for example, Harlow and Lane, Antibodies: A Laboratory Manual, Cold Spring Harbor Laboratory, NY, 1988, incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be noncovalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., 1991, In: Methods: A Companion to Methods in Enzymology, 2:97; Bird et al., 1988, Science 242:423-426; U.S. Pat. No. 4,946,778; and Pack, et al., 1993, BioTechnology 11:1271-77.

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain a minimal sequence derived from non-human immunoglobulin, such as the eitope recognising sequence. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity. Humanized antibody(es) containing a minimal sequence(s) of antibody(es) of the invention, such as a sequence(s) recognising an epitope(s) described herein, is one of the preferred embodiments of the invention.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details, see: Jones et al., 1986, Nature 321, 522-525; Reichmann et al., 1988, Nature 332, 323-329; Presta, 1992, Curr Op Struct Biol 2:593-596; Holmes et al., 1997, J Immunol 158:2192-2201 and Vaswani, et al., 1998, Annals Allergy, Asthma & Immunol 81:105-115.

The generation of antibodies may be achieved by any standard methods in the art for producing polyclonal and monoclonal antibodies using natural or recombinant fragments of a sequence selected from any of the sequences identified as SEQ ID NOs: 1-13, as an antigen. Such antibodies may be also generated using variants or fragments of SEQ ID NOs: 1-13.

The antibodies may also be produced in vivo by the individual to be treated, for example, by administering an immunogenic fragment according to the invention to said individual. Accordingly, the present invention further relates to a vaccine comprising an immunogenic fragment described above.

The application also relates to a method for producing an antibody of the invention said method comprising a step of providing of an immunogenic fragment described above.

The invention relates both to an antibody, which is capable of modulating, such as enhancing or attenuating, biological function of neuroplastin in particular a function related to neural cell growth and survival, and to an antibody, which can recognise and specifically bind to neuroplastin without modulating biological activity thereof.

The invention relates to use of the above antibodies for therapeutic applications involving the modulation of activity of neuroplastin.

In one aspect the invention relates to the use of a pharmaceutical composition comprising an antibody described above.

### REFERENCES

Bernstein H G, Smalla K H, Bogerst B, Godron-Weeks P R, Beesley P W, Gundelfinger E D, Kreutz M R, The immunolocalization of the synaptic glycoprotein neuroplastin differs substantially between the humans and the rodent brain, Brian Res., 2007, 1134:107-12.
Ditlevsen D K, Kohler L B, Pedersen M V, Risell M, Kolkova K, Meyer M, Berezin V, Bock E, The role of phosphatidylinositol 3-kinase in neural cell adhesion molecule-mediated neuronal differentiation and survival, J. Neurochem., 2003, 84:546-56.
Empson R M, Buckby L E, Kraus M, Bates K J, Crompton M R, Gundelfinger E D, Beesley P W, The cell adhesion molecule neuroplastin-65 inhibits hippocampal long-term potentiation via a mitogen-activated protein kinase p38-dependent reduction in surface expression of GluR1-containing glutamate receptors, J. Neurochem., 2006, 99: 850-60.
Ronn L C, Relets I, Hartz B P, Bech M, Berezin A, Berezin V, MØller A, Bock E, A simple procedure for qualification of neurite outgrowth based on stereological principles, J. Neurosci. Methods, 2000, 100:25-32.
Saito A, Fujikura-Ouchi Y, Kuramasu A, Shimoda K, Akiyama K, Matsuoka H, Ito C, Association study of putative polymorphisms in the neuroplastin gene and schizophrenia, Neurosci. Lett., 2007, 411: 168-73.
Schousboe A, Frandsen A, Drejer J, Evidence of evoked release of adenosine and glutamate from cultured cerebellar granule cells, Neurochem Res., 1989, 14:871-5.

### EXAMPLES

### Determination of Phosphorylation of FGFR1

Trex293 cells (Invitrogen) were stably transfected with human FGFR1, splice variant IIIc, with a C-terminal Strep II tag (IBA Biotech). The cells were maintained in Dulbecco's modified Eagle's medium with 200 μg/ml hygromycin (Invitrogen), 10% FCS, 1% glutamax, 100 U/ml penicillin, 100 ig/ml streptomycin (all from Gibco BRL, Paisley, UK). Cells were starved overnight in medium without serum before being treated with peptides. Cells were lysed in lysis buffer containing 1% Nonidet P-40 (Sigma-Aldrich, Copenhagen, Denmark), complete protease inhibitors (Roche) (1:50), phosphatase inhibitors (Calbiochem inhibitor cocktail III) (1:100) in PBS. Protein concentration was determined using the bicinchoninic acid assay (Pierce, Rockville, Ill., USA). From each lysate 500 μg protein was incubated with 15 μl agarose-coupled anti-phosphotyrosine antibodies for 6 h at 4° C. The bound proteins were washed and eluted with 180 mmol/l phenylphosphate (Sigma-Aldrich). Purified proteins (25 μl from each sample) were separated by SDS-PAGE and transferred to a polyvinylidene fluoride membrane (Millipore, Bedford, Mass., USA). Immunoblotting was performed using antibodies against the recombinant Strepll tag (IBA Biotech), developed with SuperSignal West Dura extended duration substrate (Pierce), and visualized and quantified using the SynGene Gene Tool image analysis software (Synoptics, Cambridge, UK).

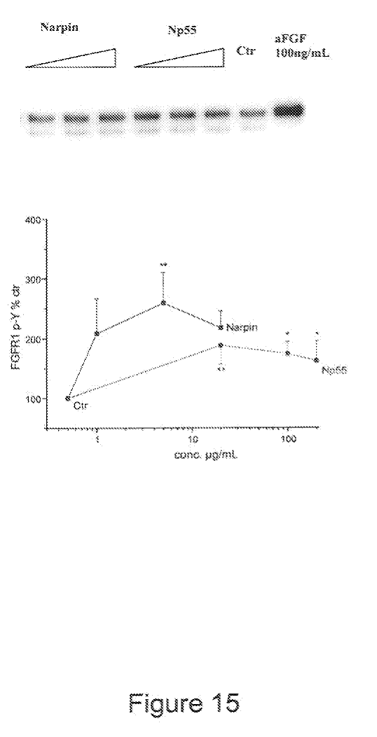

The effect of the narpin peptide (RIVTSEEVIIRDS) SEQ ID NO: 7 on FGFR Phosphorylation are shown in FIG. 15.

### Hippocampal Neurons

Hippocampal neurons were prepared from embryonic day 19 (E19) Wistar rats. A pregnant rat was killed, and the hippocampal tissue of the fetuses was dissected in ice-cold modified Krebs-Ringer solution and cleared of blood vessels and meninges. In short, the neurons were crudely homogenized by chopping before trypsin treatment and then washed in the presence of soybean trypsin inhibitor and DNAse 1 (both from Sigma) before plating in Neurobasal medium supplemented with 2% (v/v) B27, 20 mM HEPES, 1% (v/v) glutamax, 100 U/ml penicillin, and 100 Ig/ml streptomycin Invitrogen).

For neuroplastin immobilization, culture chambers were preincubated with various concentrations of the protein for 2 h at 37° C. Before cell seeding, culture chambers were washed in phosphate-buffered saline.

### Cerebellar Granule Neurons

Cerebellar granule neuron (CGN) cultures were obtained from 3-4-day-old Wistar rats (Charles River, Sulzfeld, Germany, or Moellegaard, Denmark) as previously described by Schousboe et al. (1989). Briefly, the cerebella were dissected, cleared of meninges and blood vessels, chopped, and trypsinized. The neurons were washed in the presence of DNAse 1 and soybean trypsin inhibitor (Sigma), and cellular debris was pelleted by centrifugation; the cells resuspended and then plated on poly-L-lysine (PLL; Sigma)-coated or uncoated microtiter plates in Neurobasal medium supplemented with 4% (w/v) bovine serum albumin (BSA), 2% (v/v) B27, 1% (v/v) glutamax, 100 U/ml penicillin, 100_g/ml streptomycin, 4.5 g D-glucose/L (Sigma) 0.25% (v/v) sodium pyruvate, and 2% (v/v) 1 M HEPES (Gibco BRL) for neurite outgrowth assays; in Neurobasal-A medium supplemented penicillin, 100 μg/ml streptomycin (Gibco BRL), and a final concentration of 40 mM KCl for survival assays; or in Neurobasal-A medium supplemented with 0.5% (v/v) glutamax, 100 U/ml penicillin, 100 μg/ml streptomycin for the PACE assay.

### Neurite Outgrowth Assay

Dissociated hippocampal neurons (or CGN) were plated at a density of 12,000 cells/cm2 on plastic in eight-well Permanox Lab-Tek chamber slides in Neurobasal medium supplemented as described above. Twenty-four hours later, the cells were fixed in 4% w/v paraformaldehyde for 20 min, stained with Coomassie Blue R250 (4 g/liter in 45% v/v ethanol and 45% v/v acetic acid) for 20 min, Images of at least 200 cells were grabbed for each group in each individual experiment in a systematic series of fields of view as previously described (Ronn et al., 2000) by computer-assisted fluorescence microscopy with a Nikon Plan 320 objective (Nikon, Tokyo, Japan) and a video camera (Grundig Electronics). The average neurite length per cell was estimated by using a stereological approach (Ronn et al., 2000) and the software package Prima developed at the Protein Laboratory (Copenhagen, Denmark).

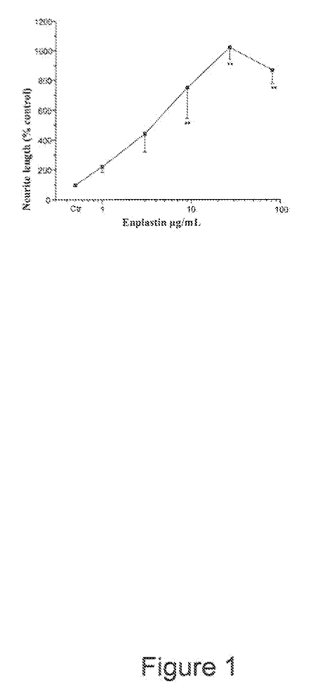

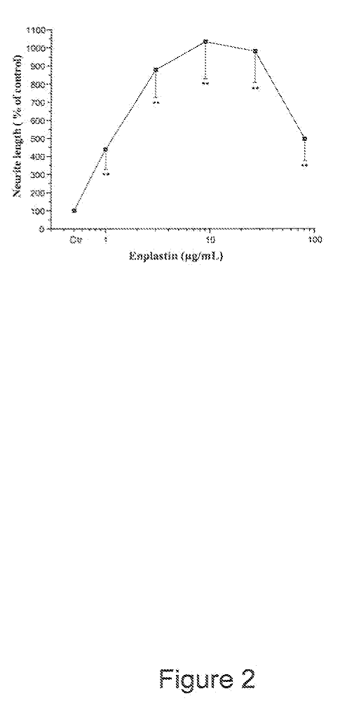

The effect of the Enplastin peptide (DPKRNDLRQNPSITWIR) SEQ ID NO: 6 on neurite outgrowth from cerebellar granule neurons (CGN) and hippocampal neurons from postnatal day 7 rats are shown in FIGS. 1 and 2 respectively.

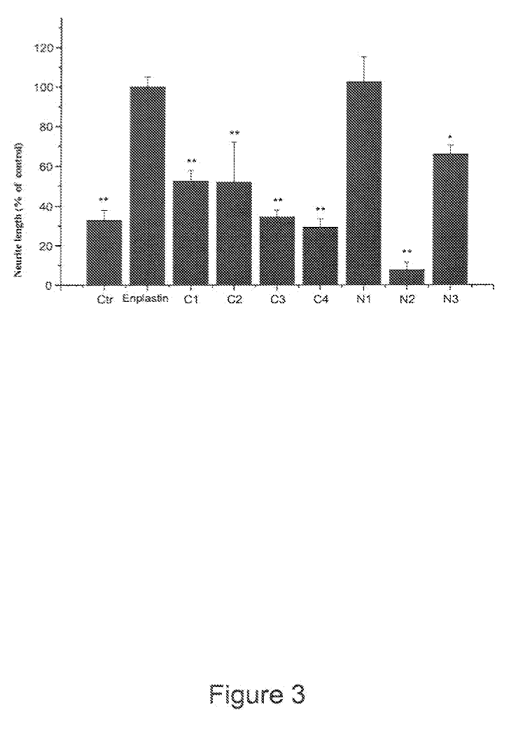

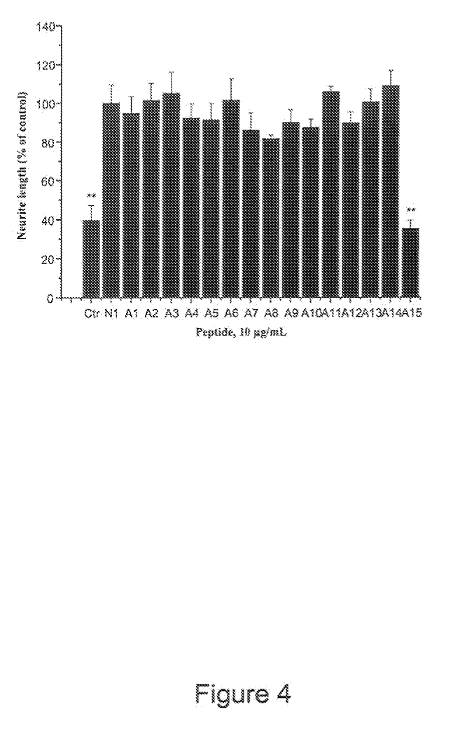

The effect of the truncated versions of Enplastin peptide (DPKRNDLRQNPSITWIR), 10 μg/ml each on neurite outgrowth from cerebellar granule neurons (CGN) from postnatal day 7 rats are shown in FIG. 3. The effect of the alanine substitution of the N1-truncated Enplastin peptide (DPKRNDLRQNPSITWIR) SEQ ID NO: 6 on neurite outgrowth from cerebellar granule neurons (CGN) from postnatal day 7 rats are shown in FIG. 4.

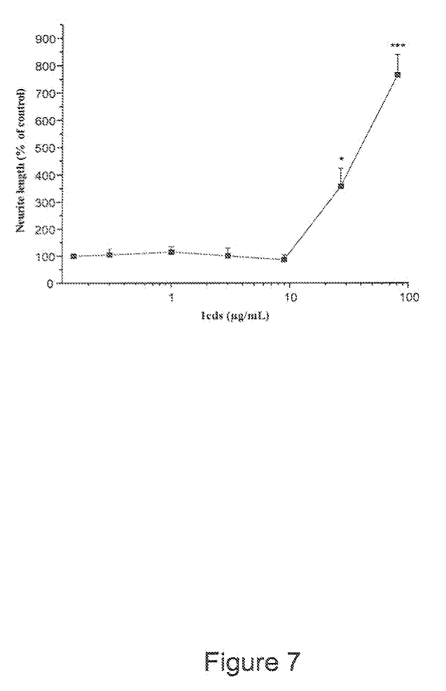

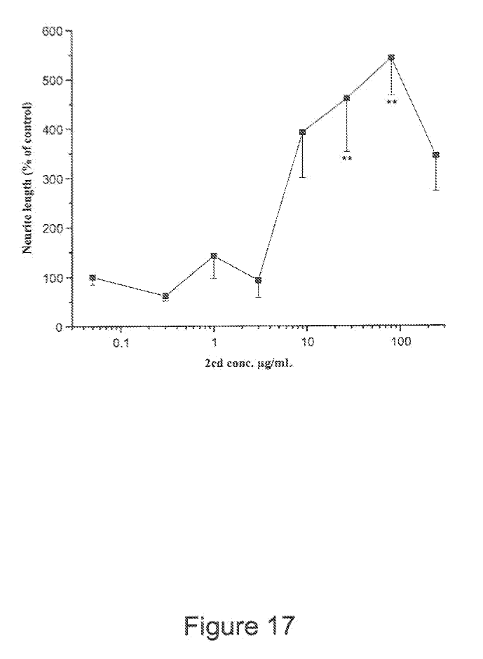

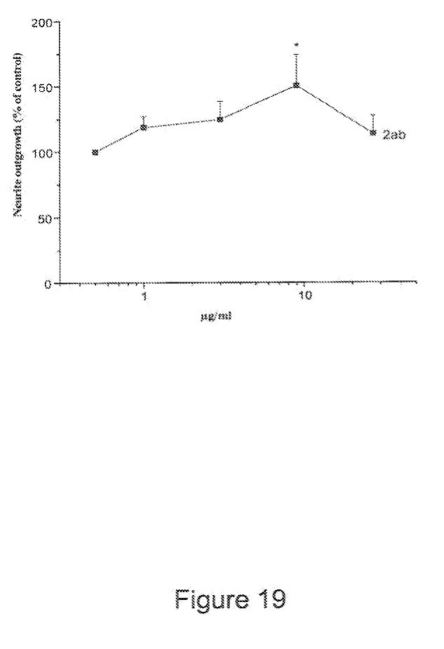

The effects of the 1 cds peptide (NRAESFRQLWDGAR) SEQ ID NO: 4, the 2 cd peptide (TKNGVELTATRKNA) SEQ ID NO: 9, and the narpin peptide (RIVTSEEVIIRDS) SEQ ID NO: 7, on neurite outgrowth from cerebellar granule neurons (CGN) from postnatal day 7 rats are shown in FIGS. 7, 17, and 19, respectively.

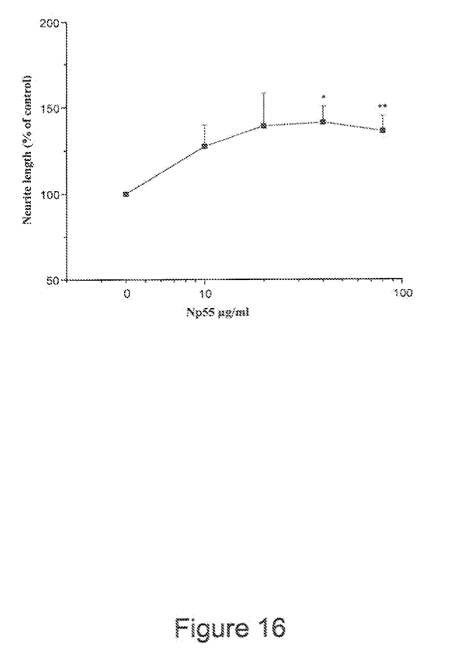

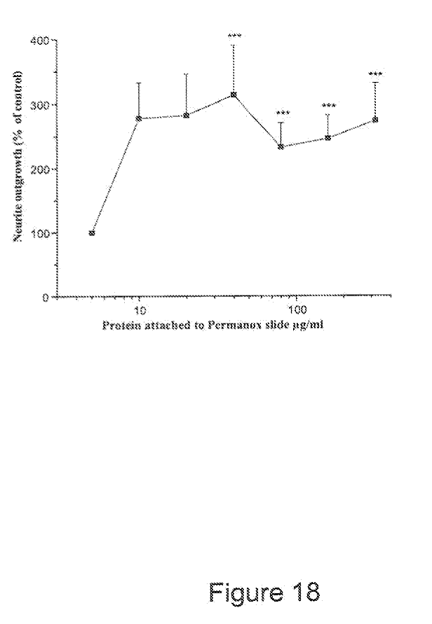

The effects of the Neuroplastin 55 ectodomain in solution and of the Neuroplastin 55 ectodomain, coated on the slide, on neurite outgrowth from cerebellar granule neurons (CGN) are shown in FIGS. 16 and 18, respectively.

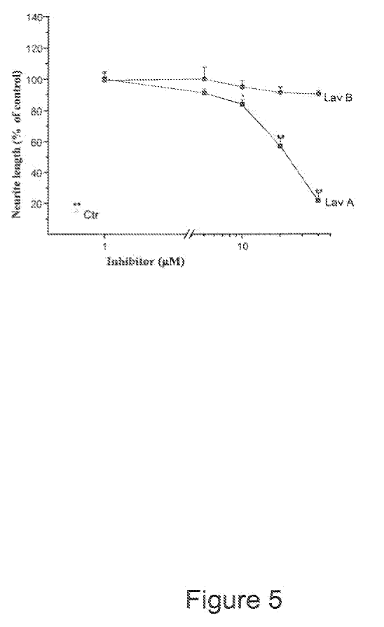

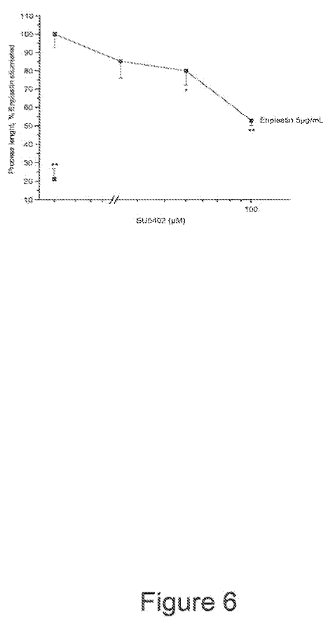

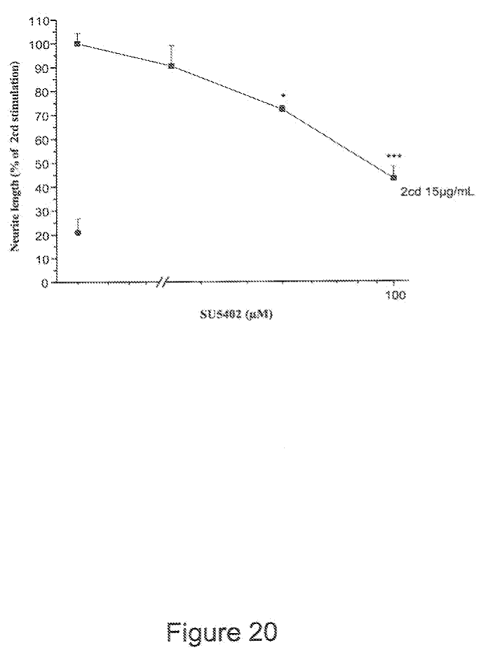

The effects of the inhibitor of receptor tyrosine kinases (RTK) (lavendustin A) on Enplastin-induced (DPKRNDLRQNPSITWIR, SEQ ID NO: 6) neurite outgrowth from CGN are shown in FIG. 5. Lavendustin B is a control, which does not inhibit RTK. The effect of the inhibitor of FGFR (SU5402) on Enplastin-induced neurite outgrowth (DPKRNDLRQNPSITWIR) SEQ ID NO: 6 is shown In FIG. 6. The effect of the inhibitor of FGFR, SK5402 on 2 cd-induced neurite outgrowth from hippocampal neurons are shown in FIG. 20.

### PACE

Phosphospecific antibody cell-based ELISA assay (PACE) Phosphorylated PKC in hippocampal neurons was detected according to Versteeg et al. (2000). Briefly, hippocampal neurons were plated at a density of 100 000 neurons/well in a 96-well microtiter plate (Nunc) directly in serum free basal modified Eagle's medium (Gibco-BRL) supplemented with 1% (v/v) glutamax, 3.5 g D-glucose/L, 1% (v/v) sodium pyruvate (Gibco-BRL), 100 Ig/mL streptomycin and 5 mM KCl. Neurons were stimulated 4 h later with different peptides for 10 min to determine Akt-p or 30 min to determine Erk-p or CREB-p. bFGF was used as a positive control.

The plates were centrifuged at 70 g for 10 min, fixed in 4% (v/v) formaldehyde and stained using polyclonal antibodies against phospho-PKC (pan) and peroxidase-conjugated secondary antibodies. A450 was measured and the staining intensity was normalized to the total number of cells, which was estimated by staining with crystal violet followed by absorbance measurement at A600.

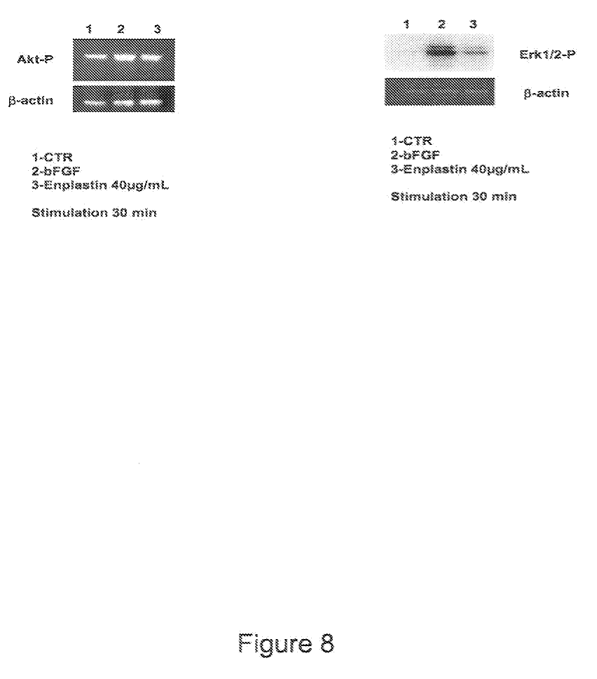

The effect of the Enplastin peptide (DPKRNDLRQNPSITWIR) SEQ ID NO: 6 on Akt (A) and Erk1/2 B phosforylation in primary hippocampal neurons is shown in FIG. 8. Cells were grown for 6 h and then treated with 40 μg/mL Enplastin or 10 nM FGF2 for 10 min (Akt) or 30 min (Erk) and further immunoblotted for phospho-p44/p42 Map Kinase (Thr202/Tyr204) or Ser473-phosphorylated Akt. Representative immunoblots are shown, 1. control. 2. GFGF (10 μM). 3. Enplastin (40 μg/ml).

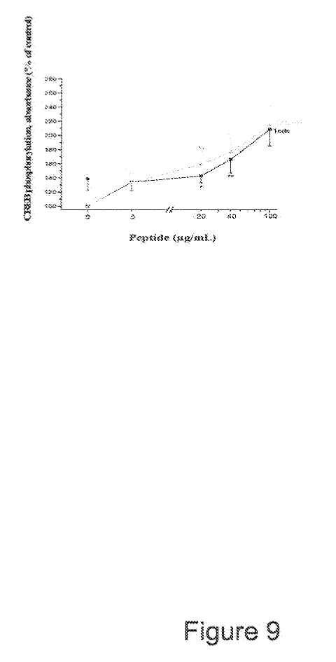

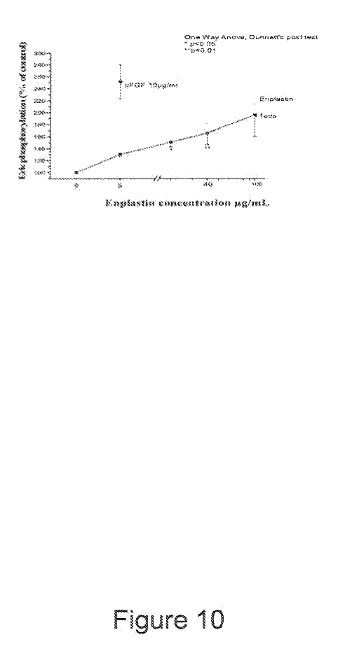

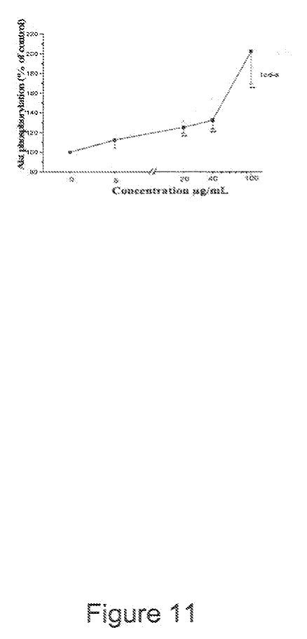

The effects of the Enplastin peptide (DPKRNDLRQNPSITWIR) SEQ ID NO: 6 and the 1 cds peptide (NRAESFRQLWDGAR) SEQ ID NO: 4 on CREB phosphorylation in primary cultures of hippocampal neurons are shown in FIG. 9. Hippocampal neurons were grown in serum-free medium for 4 h and then the cells were treated with 5, 20, 40 and 100 μg/mL Enplastin 1 fg or 1 cds for 30-min. Phosphorylated CREB was identified by phospho-CREB specific antibodies, and total cell number was estimated by crystal violet staining. The effects of the Enplastin peptide (DPKRNDLRQNPSITWIR) SEQ ID NO: 6 and the 1 cds peptide (NRAESFRQLWDGAR) SEQ ID NO: 4 on Erk phosphorylation in primary cultures of hippocampal neurons are shown in FIG. 10. Hippocampal neurons were grown in serum-free medium for 4 h and then the cells were treated with 5, 20, 40 and 100 μg/mL Enplastin or 1 cds for 30-min. Phosphorylated Erk was identified by phospho-Erk specific antibodies, and total cell number was estimated by crystal violet staining. The effects of the Enplastin peptide (DPKRNDLRQNPSITWIR) SEQ ID NO: 6 and 1 cds peptide (NRAESFRQLWDGAR) SEQ ID NO: 4 on Akt phosphorylation in primary cultures of hippocampal neurons are shown in FIG. 11. Primary cultures of hippocampal neurons were grown in serum-free medium for 4 h and then the cells were treated with 5, 20, 40 and 100 μg/mL Enplastin or 1 cds for 30-min. Phosphorylated Akt was identified by phospho-Akt specific antibodies, and total cell number was estimated by crystal violet staining.

### SPR Analysis

Binding analysis were performed using a BIAcore2000 instrument (Biosensor AB, Sweden) at 25° C. using 10 mM sodium phosphate containing 150 mM NaCl (pH 7.4) as running buffer. Data were analyzed by nonlinear curve fitting using the manufacturer's software. The FGFR1, Narpin and reversed Narpin were immobilized on a sensor chip. The binding is expressed in resonance units and corresponds to the difference in binding to the flow cell with immobilized protein and a reference flow cell. Peptides were injected at concentrations 50 and 100 μg/ml peptide in HBS-EP buffer at flow rate 20 μl/min.

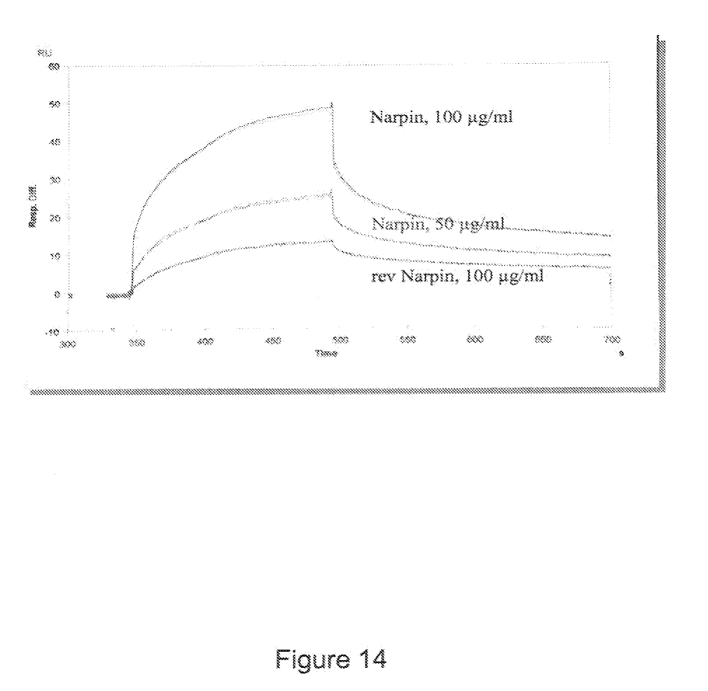

The tested peptides (FIG. 14) were:
a) Narpin, RIVTSEEVIIRDS (SEQ ID NO: 7)
b) Reversed narpin, SDRIIVEETVIR (SEQ ID NO: 36)

### Intracellular [Ca2+]

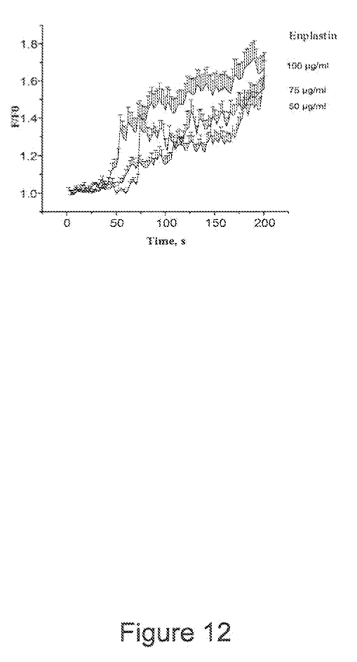

The dose-response relationship for the Enplastin peptide SEQ ID NO: 6 (DPKRNDLRQNPSITWIR)-triggered [Ca2+] rise in hippocampal neurons are shown in FIG. 12.

### Survival Assay

Primary cultures of CGN were plated at a density of 100 000 cells/cm2 on poly-L-lysine coated 8-well permanox slides in Neurobasal-A medium (Gibco BRL) supplemented with 2% (v/v) B27, 0.5% (v/v) glutamax, 100 units/mL penicillin, 100 Ig/mL streptomycin and KCl, making the final concentration of KCl in the medium 40 mM. Twenty-four hours after plating, cytosine-b-D-arabinofuranoside (Ara-C; Sigma-Aldrich) was added to a final concentration of 10 IM to avoid proliferation of glial cells, after which the neurons were allowed to differentiate for a further 6 days at 37_C. Apoptotic cell death was induced by washing twice and changing the medium to Basal Medium Eagle (BME; Gibco BRL) supplemented with 1% (v/v) glutamine, 100 U/mL penicillin and 100 Ig/mL streptomycin, 3.5 g D-glucose/L and 1% (v/v) sodium pyruvate (Gibco BRL) together with various concentrations of peptide. Thereby the concentration of potassium in the cultures was reduced to 5 mM KCl (Ditlevsen et al., 2003). Two days after induction of apoptosis, the cells were fixed with 4% (v/v) formaldehyde and stained with Hoechst 33258 as described for the survival assay employing hippocampal neurons.

1. A pharmaceutical composition comprising a peptide comprising an amino acid sequence of neuroplastin (SEQ ID NO:1) or a fragment or variant thereof, said peptide being capable of modulating the activity of receptor tyrosine kinases.

2. The pharmaceutical composition according to claim 1, wherein said peptide or variant is capable of modulating the activity of FGFRs.

3. The pharmaceutical composition according to any of the preceding claims, wherein said peptide comprises one or more of the following amino acid sequences:

| | |
|---|---|
| TKLTGDAFEL | SEQ ID NO: 2 |
| DVVGSPTPEIQ | SEQ ID NO: 3 |
| NRAESFRQLWDGAR | SEQ ID NO: 4 |
| RRVTVNTAYGSNG | SEQ ID NO: 5 |
| DPKRNDLRQNPSITWIR | SEQ ID NO: 6 |
| RIVTSEEVIIRDS | SEQ ID NO: 7 |
| NLTSSSHTLMYS | SEQ ID NO: 8 |
| TKNGVELTATRKNA | SEQ ID NO: 9 |
| KNASNMEYRINKP | SEQ ID NO: 10 |
| NKPRAEDSGE | SEQ ID NO: 11 |
| VYHFVSAPKANAT | SEQ ID NO: 12 |
| INKENYTELN | SEQ ID NO: 13 |

or a fragment, or variant thereof.

4. The pharmaceutical composition according to claim 3, wherein said peptide fragment is a variant fragment, wherein said variant sequence is at least 75% identical to said fragment, such as wherein the variant sequence is at least 80% identical to said fragment, such as wherein the variant sequence is at least 90% identical to said fragment.

5. The pharmaceutical composition according to any of claims 1-3, wherein the peptide comprises the amino acid sequence TKLTGDAFEL (SEQ ID NO:2).

6. The pharmaceutical composition according to any of claims 1-3, wherein the peptide comprises the amino acid sequence DVVGSPTPEIQ (SEQ ID NO:3).

7. The pharmaceutical composition according to any of claims 1-3, wherein the peptide comprises the amino acid sequence NRAESFRQLWDGAR (SEQ ID NO:4).

8. The pharmaceutical composition according to any of claims 1-3, wherein the peptide comprises the amino acid sequence RRVTVNTAYGSNG (SEQ ID NO:5).

9. The pharmaceutical composition according any of claims 1-3, wherein the peptide comprises the amino acid sequence DPKRNDLRQNPSITWIR (SEQ ID NO:6).

10. The pharmaceutical composition according to any of claims 1-3, wherein the peptide comprises the amino acid sequence RIVTSEEVIIRDS (SEQ ID NO:7).

11. The pharmaceutical composition according to any of claims 1-3, wherein the peptide comprises the amino acid sequence NLTSSSHTLMYS (SEQ ID NO:8).

12. The pharmaceutical composition according to any of claims 1-3, wherein the peptide comprises the amino acid sequence TKNGVELTATRKNA (SEQ ID NO:9).

13. The pharmaceutical composition according to any of claims 1-3, wherein the peptide comprises the amino acid sequence KNASNMEYRINKP (SEQ ID NO:10).

14. The pharmaceutical composition according to any of claims 1-3, wherein the peptide comprises the amino acid sequence NKPRAEDSGE (SEQ ID NO:11).

15. The pharmaceutical composition according to any of claims 1-3, wherein the peptide comprises the amino acid sequence VYHFVSAPKANAT (SEQ ID NO:12).

16. The pharmaceutical composition according to any of claims 1-3, wherein the peptide comprises the amino acid sequence INKENYTELN (SEQ ID NO:13).

17. The pharmaceutical composition according to any of the preceding claims, wherein said peptide is capable of modulating Akt activity.

18. The pharmaceutical composition according to any of the preceding claims, wherein said peptide is capable of modulating Erk 1/2 activity.

19. The pharmaceutical composition according to any of the preceding claims, wherein said peptide is capable of modulating CREB activation.

20. The pharmaceutical composition according to any of the preceding claims, wherein said peptide is capable of stimulating neurite outgrowth.

21. The pharmaceutical composition according to any of the preceding claims, wherein said peptide is capable of stimulating cell survival.

22. The pharmaceutical composition according to any of the preceding claims, wherein said peptide is capable of stimulating synaptic plasticity.

23. The pharmaceutical composition according to any of the preceding claims, wherein said peptide is capable of stimulation learning and/or memory.

24. The pharmaceutical composition according to any of the preceding claims used for the production of a medicament for treatment of diseases or conditions wherein modulation of receptor tyrosine kinases is essential.

25. The pharmaceutical composition according to any of the preceding claims used for the production of a medicament for treatment of diseases or conditions wherein modulation of FGFRs is essential.

26. The pharmaceutical composition according to any of the preceding claims used for the production of a medicament for treatment of diseases or conditions of the central or peripheral nervous system.

27. The pharmaceutical composition according to any of the preceding claims used for the production of a medicament for treatment of a disease or condition wherein stimulation of neural cell differentiation, neural cell survival, neurogenesis, stem cell proliferation, stem cell differentiation, and/or learning and memory is beneficial for recovery from said disease or condition.

28. The pharmaceutical composition according to any of the preceding claims used for the production of a medicament for treatment of postoperative nerve damage, traumatic nerve damage, impaired myelination of nerve fibers, postischaemic damage, multiinfarct dementia, multiple sclerosis, nerve degeneration associated with diabetes mellitus, neuro-muscular degeneration, schizophrenia, mood disorders, manic depressive disorders, Alzheimer's disease, Parkinson's disease, or Huntington's disease.

29. The pharmaceutical composition according to any of the preceding claims used for the production of a medicament for treatment of diseases or conditions of the muscles including conditions with impaired function of neuro-muscular connections, or for the treatment of diseases or degenerative conditions of the gonads, pancreas or kidney.

30. The pharmaceutical composition according to any of the preceding claims used for the production of a medicament capable of preventing cell death of heart muscle cells.

31. The pharmaceutical composition according to any of the preceding claims used for the production of a medicament capable of stimulating revascularisation.

32. The pharmaceutical composition according to any of the preceding claims used for the production of a medicament capable of promotion of wound healing.

33. The pharmaceutical composition according to any of the preceding claims used for the production of a medicament capable of inhibiting angiogenesis.

34. The pharmaceutical composition according to any of the preceding claims used for the production of a medicament for treatment of cancer.

35. The pharmaceutical composition according to any of the preceding claims used for the production of a medicament capable of stimulation of the ability to learn and/or of the short and/or long term memory.

36. The pharmaceutical composition according to any of the preceding claims used for the production of a medicament capable of modulating proliferation and/or differentiation and/or regeneration and/or morphological plasticity of cells.

37. A peptide being a variant or a fragment of neuroplastin (SEQ ID NO:1), wherein said variant or fragment is as defined in any of claims 1-16.

38. The peptide according to claim 37, wherein said peptide is capable of modulating Akt activity.

39. The peptide according to claim 37, wherein said peptide is capable of modulating Erk 1/2 activity.

40. The peptide according to claim 37, wherein said peptide is capable of modulating CREB activation.

41. The peptide according to claim 37, wherein said peptide is capable of stimulating neurite outgrowth.

42. The peptide according to claim 37, wherein said peptide is capable of stimulating cell survival.

43. The peptide according to claim 37, wherein said peptide is capable of stimulating synaptic plasticity.

44. The peptide according to claim 37, wherein said peptide is capable of stimulation learning and/or memory.

45. The peptide as defined in any of claims 37-44 for use as a medicament.

46. The peptide as defined in any of claims 37-44 for the treatment of any of the conditions or diseases as defined in any of claims 24-36.

47. Use of a peptide as defined in claim 37 for the production of an antibody.

48. An antibody capable of binding to an epitope comprising a sequence corresponding to neuroplastin or a fragment thereof or a variant.

49. An antibody capable of binding to an epitope comprising at least one of the following sequences:

| | |
|---|---|
| TKLTGDAFEL | SEQ ID NO: 2 |
| DVVGSPTPEIQ | SEQ ID NO: 3 |
| NRAESFRQLWDGAR | SEQ ID NO: 4 |
| RRVTVNTAYGSNG | SEQ ID NO: 5 |
| DPKRNDLRQNPSITWIR | SEQ ID NO: 6 |
| RIVTSEEVIIRDS | SEQ ID NO: 7 |
| NLTSSSHTLMYS | SEQ ID NO: 8 |
| TKNGVELTATRKNA | SEQ ID NO: 9 |
| KNASNMEYRINKP | SEQ ID NO: 10 |
| NKPRAEDSGE | SEQ ID NO: 11 |
| VYHFVSAPKANAT | SEQ ID NO: 12 |
| INKENYTELN. | SEQ ID NO: 13 |

50. The antibody according to claim 41, wherein said antibody is capable of modulating biological activity mediated by receptor tyrosine kinase.

51. The antibody according to claim 41, wherein said antibody is capable of modulating biological activity mediated by FGFRs.

52. The antibody according to claim 41, wherein said antibody is capable of modulating biological activity mediated by Akt.

53. The antibody according to claim 41, wherein said antibody is capable of modulating biological activity mediated by Erk 1/2.

54. The antibody according to claim 41, wherein said antibody is capable of modulating biological activity mediated by CREB.

55. Use of an antibody according to claims 41-47 for the manufacture of a medicament for treatment of conditions or disesases as defined in claims 17-36.

56. A pharmaceutical composition comprising an antibody according to any of claims 41-45.
